(19) United States
(12) Patent Application Publication
(10) Pub. No.: US 2007/0218002 A1
(43) Pub. Date: Sep. 20, 2007

(54) Method for Detecting Alzheimer's Disease and other Forms of Dementia, and Measuring Their Progression

(76) Inventors: Jorge R. Barrio, Agoura Hills, CA (US); Vladimir Kepe, Los Angeles, CA (US); Gary W. Small, Los Angeles, CA (US)

Correspondence Address: CHRISTIE, PARKER & HALE, LLP, PO BOX 7068, PASADENA, CA, 91109-7068, US

(21) Appl. No.: 11/579,763

(22) PCT Filed: May 9, 2005
(86) PCT No.: PCT/US05/16061

§ 371 Date: Nov. 7, 2006

Related U.S. Application Data
(60) Provisional application No. 60/569,136, filed on May 7, 2004.

(51) Int. Cl. A61K 49/00; G01N 33/543

(52) U.S. Cl. 424/9.1

(57) ABSTRACT

The invention provides a method for detecting or monitoring Alzheimer's disease and other forms of dementia using positron emission tomography (PET) or single-photon emission computed tomagraphy (SPECT) and radiolabeled, serotonin 5-HT_{1A} receptor-specific tracers (such as [F-18]MPPF, [F-18]FCWAY, [C-11]WAY-100635, and other radiolabeled compounds having agonistic or antagonistic effect on serotonin receptors), for detection or monitoring of pathological changes (i.e., neuronal cell loss) associated with dementia.

### CROSS-REFERENCE TO RELATED APPLICATION

This application is based on and claims priority of U.S. Provisional Application No. 60/569,136, filed May 7, 2004, the entire disclosure of which is incorporated by reference herein.

### ACKNOWLEDGEMENT OF GOVERNMENT SUPPORT

This invention was made with Government support, Grant No. DE-FC03-02ER63420, awarded by the Department of Energy. The Government has certain rights in the invention.

### FIELD OF THE INVENTION

The invention relates generally to methods for diagnosing and assessing alzheimer's disease and other forms of dementia.

### BACKGROUND OF THE INVENTION

Alzheimer's disease (AD) is a neurodegenerative disease causing neuronal cell death in selected vulnerable populations of neurons, and disconnection of cortico-cortical and cortico-hippocampal brain circuits responsible for memory and cognition. (Related diseases include frontal lobe dementia and Lewy body dementia.) Currently, there is no reliable method for direct, in vivo detection of neuronal cell loss in patients. MRI-based morphometry measuring cortical atrophy has been used to assess progression of Alzheimer's disease, but it is an indirect measurement technique: it measures the volumes of brain tissue and not any of the neuronal cell features. There is great interest in the medical field to develop reliable, non-invasive diagnostic measures for early detection and on-going monitoring of Alzheimer's disease and other types of dementia through direct, in vivo quantitation of neuronal cell loss.

The medial temporal lobe is the place of earliest pathological changes caused by the disease, including substantial loss of large pyramidal neurons in the CA fields of the hippocampus and in the subiculum. (Braak and Braak, 1991; Delacourte et al., 1999; De Lacoste et al., 1993; Morrison and Hof, 2002)

Large pyramidal neurons in hippocampal formation are glutamatergic neurons using excitatory amino acids—glutamic acid and aspartatic acid—as neurotransmitters. Although these neuronal cells communicate via glutamatergic neurotransmission, and use a variety of glutamatergic receptors for that purpose, they also receive modulatory input via other types of receptors (acetylcholine, serotonin, corticosteroids, etc.) expressed on different areas of the neuron. These receptors either increase or decrease hyperpolarization and, in this way, activate or deactivate the neuron for its primary function: relay of signal via glutamatergic neurotransmission. These pyramidal neurons contain only one type of serotoninergic heteroreceptor: the 1A subtype of serotonin receptors with high affinity for serotonin (K_{D}=˜3 nM). (Morrison and Hof, 2002; Vizi and Kiss, 1998)

The serotoninergic neuronal cells projecting to the hippocampal formation pyramidal neurons are located in the dorsal raphe nucleus. They also express serotonin 1A (5-HT_{1A}) receptors on their cell bodies and modulate their own activity via these receptors. The serotoninergic projections reaching the hippocampus do not form synapses with the pyramidal neuronal cells; serotonin is released free in the area of pyramidal neurons and reaches 5-HT_{1A} receptors by diffusion. For that reason, the pyramidal cells have very high levels of 5-HT_{1A} receptors expressed on the axons proximal to the cell bodies in order to compensate for the low level of serotonin available. (Azmitia et al., 1996)

The loss of these large pyramidal neurons also means loss of 5-HT_{1A} receptors (in proportionate or disproportionate fashion, as the remaining functional neurons may compensate the loss), as demonstrated with in vitro binding experiments ([3H]MPPF and [3H]8-OH-DPAT) in animal models of neuronal cell loss caused by neurotoxins kainic acid or volkensin. (van Bogaert et al., 2001; Francis et al., 1992; Bowen et al., 1993)

The decrease of 5-HT_{1A} concentration in different areas of the brains from Alzheimer's disease patients has been demonstrated in vitro either by autoradiography or by binding experiments with [3H]8-OH-DPAT. (Palmer et al., 1987, Middlemiss et al., 1986)

In summary, it has been observed that the hippocampal formation is affected early in the Alzheimer's disease progression; the disease causes significant loss of large pyramidal neurons in hippocampus and elsewhere in the cortex; and these large pyramidal neurons have very high level of 5-HT_{1A} receptors expressed on the axons proximal to soma, with various concentration (Bₘₐₓ) throughout the neocortex, with the maximum level in the hippocampus. What is needed is a reliable method for direct, in vivo detection of neuronal cell loss in patients suffering from Alzheimer's disease and other forms of dementia, such as frontal lobe dementia and Lewy body dementia.

Positron emission tomography (PET) is a technique that allows in vivo measurements of brain receptor concentrations (Bₘₐₓ) in living humans. Radiolabeled compounds (radiolabeled with positron-emitting isotopes, e.g., [F-18]fluorine (t_{1/2}=110 min) or [C-11]carbon (t_{1/2}=20 min)) having high specificity for the particular receptor, and having high affinity (in nM range), are needed for such measurements.

A variety of radiolabeled compounds with either agonistic or antagonistic effect on 5-HT_{1A} receptors has been developed. (Passchier and van Waarde, 2001) The antagonists include several compounds that have been tested for use with PET and that have proven to be useful for the in vivo quantitation of brain 5-HT_{1A} receptor densities. These compounds are [F-18]fluorine labeled MPPF (Passchier et al., 2001; Passchier et al., 2000) [F-18]FCWAY, and [C-11]carbon-labeled WAY100635. These compounds have been tested in healthy volunteers, and comparison of results of the [F-18]MPPF and [C-11]carbonyl-WAY100635 experiments shows linear correlation. (Passchier et al., 2000) They have also been used for PET imaging of depression, anxiety, schizophrenia, panic disorder. (Dreverts et al., 2000; Tauscher et al., 2002; Neumeister et al., 2004; Cidis Meltzer et al., 2001;) All of these disorders are known to show some level of brain serotoninergic system dysfunction; none of these disorders shows any significant pyramidal neuronal loss. [F-18]MPPF and [C-11]carbon labeled [C-11]carbonyl-WAY100635 have also been utilized for the studies of temporal lobe epilepsy in which neuronal loss is predicted. (Merlet et al., 2004; Toczek et al., 2003)

All previous uses compared the measured the densities of serotonin 5-HT_{1A} receptors as a result of psychiatric conditions (depression, panic disorders, . . . ). The effect of other drugs (e.g. pindolol) on 5-HT_{1A} receptors has also been reported. Until the present invention, however, no one has used PET to determine the decrease of serotonin 5-HT_{1A} receptors as a result of cell loss, in vivo, and thereby diagnose and/or monitor Alzheimer's disease and other forms of dementia.

### SUMMARY OF THE INVENTION

The present invention addresses the long-felt need for an in vivo, quantitative assessment of neuronal cell loss associated with Alzheimer's disease (“AD”) and related forms of dementia, including frontal lobe dementia and Lewy body dementia. According to a first aspect of the invention, a method for detecting or monitoring neuronal cell loss associated with dementia in a subject, in vivo, is provided, and comprises administering a radiolabeled, serotonin 5-HT_{1A} receptor-specific tracer to the subject; creating at least one image of the subject's brain using positron emission tomography (PET) or single-photon emission computed tomography (SPECT); quantitating serotonin 5-HT_{1A} receptor density in an imaged region of the subject's brain; and assessing neuronal cell loss associated with dementia by comparing the at least one image to a control or a prior image of the subject's brain. The method is useful in evaluating subjects known to suffer from AD, subjects who are suspected to suffer from AD (for example, subjects exhibiting mild cognitive impairment), and subjects who are, or appear to be, cognitively normal.

In a second aspect of the invention, a method of quantitatively evaluating neuronal cell loss associated with dementia in a subject, in vivo, is provided and comprises (a) administering a radiolabeled, 5-HT_{1A} receptor-specific tracer to the subject; (b) using PET or SPECT to generate a dynamic data set corresponding to radioactivity in the subject's brain; (c) generating a parametric data set from the dynamic data set; (d) identifying a set of regions-of-interest in the subject's brain; (e) using the parametric data set to determine tracer binding potential values for the set of regions-of-interest; and (f) comparing the determined tracer binding potential values with tracer binding potential values obtained from (i) a prior PET or SPECT scan of the subject, or (ii) a PET or SPECT scan of an age-matched, cognitively normal control. In some embodiments, steps (a)-(f) are repeated two or more times.

In a third aspect of the invention, a method of quantitatively monitoring neuronal cell loss, in vivo, in a subject known or suspected to be suffering from dementia is provided, and comprises (a) administering a radiolabeled, 5-HT_{1A} receptor-specific tracer to the subject; (b) using PET or SPECT to generate a dynamic data set corresponding to radioactivity in the subject's brain; (c) generating a parametric data set from the dynamic data set; (d) identifying a set of regions-of-interest in the subject's brain; (e) using the parametric data set to determine tracer binding potential values for the set of regions-of-interest; and (f) comparing the determined tracer binding potential values with tracer binding potential values obtained from a prior PET or SPECT scan of the subject.

In another aspect of the invention, a method for detecting or monitoring Alzheimer's disease in a subject is provided, and comprises: administering a radiolabeled, serotonin 5-HT_{1A} receptor-specific tracer to the subject; creating at least one image of the subject's brain using PET or SPECT; quantitating serotonin 5-HT_{1A} receptor density in an imaged region of the subject's brain; and assessing existence or progression of Alzheimer's disease in the subject by comparing the image(s) to a control or a prior image of the subject's brain.

While many details and alternatives are provide in the description that follows, in one embodiment, the invention can be characterized as follows: Neuronal cell loss is detected by quantitating serotonin ⁵-HT_{1A} receptor density or serotonin 5-HT_{1A} receptor total number in an imaged region of the brain. An acquired data set is reconstructed and attenuation corrected. The generated image files are analyzed by the means of Logan plot graphical analysis with the cerebellum as the reference region, and parametric images are generated. (Alternatively, tracer kinetic modeling is used.) Every voxel is represented by a parameter, tracer binding potential (BP), which is in direct correlation with 5-HT_{1A} receptor concentration in that voxel (BP=Bₘₐₓ/K_{D}). Multiplication of the BP value for a specific region of interest with its volume results in the value for total amount of serotonin 5-HT_{1A} receptors in the analyzed region of interest. Comparison of the data on a voxel by voxel basis (SPM analysis, NEUROSTAT analysis) or by comparing manually drawn regions of interest is then used to measure the BPs in AD subjects and compare them with cognitively normal, age-matched controls.

### BRIEF DESCRIPTION OF THE DRAWINGS

The invention will become better understood when considered in conjunction with the appended drawings, wherein:

FIGS. 1(A)-1(C) are plots showing the group distribution of [F-18]MPPF hippocampus BP values (1A), [F-18]MPPF hippocampus BPT values (1B), and hippocampus volume values (1C) for controls (blue), MCIs (yellow), and ADs (red); hippocampus volume is given in cm³; in all three cases controls are statistically significantly separated from MCIs (P<0.05) and from ADs (P<0.001).

FIGS. 2(A) and 2(B) are scatter plots showing the correlation between hippocampus volume and hippocampus [F-18]MPPF BP (2A; Spearman's r=0.76, P=0.0002), and between hippocampus volume and hippocampus [F-18]MPPF BPT (2B; Spearman's r=0.95, P<0.0001) for controls (blue), MCIs (yellow), and ADs (red); hippocampus volume is given in cm³.

FIGS. 3(A) and 3(B) are scatter plots correlating cognitive performance scores (MMSE scores) with hippocampus [F-18]MPPF BPT (3A; Spearman's r=0.67, P=0.0015), and MMSE scores with hippocampus [F-18]MPPF BP (3B; Spearman's r=0.82, P<0.0001) for controls (blue), MCIs (yellow), and ADs (red); the outlaying AD case (*) is the presenilin-2 mutation patient.

FIGS. 4(A)-4(F) are scatter plots showing positive hippocampus [F-18]MPPF BPT values correlation with [F-18]FDG uptake measures: average parietotemporal (global) SUVR (4A; Spearman's r=0.80, P<0.0001), posterior cingulate gyrus SUVR (4B; Spearman's r=0.73, P=0.0004), and medial temporal lobe SUVR (4C; Spearman's r=0.71, P<0.0001); in contrast, negative correlation of hippocampus [F-18]MPPF BPT values with [F-18]FDDNP binding measures were observed: global DVR (4D; Spearman's r=−0.86, P<0.0001), posterior cingulate gyrus DVR (4E; Spearman's r=−0.71, P=0.0007), and medial temporal lobe DVR (4F; Spearman's r=−0.61, P=0.006); controls (blue), MCIs (yellow), and ADs (red).

FIG. 5. is a set of brain PET images from a control subject (left column) and an AD patient (right column): [F-18]MPPF (upper row), [F-18]FDG (middle row), and [F-18]FDDNP (lower row); the images from the AD patient show strongly decrease level of [F-18]MPPF binding in hippocampus coinciding with decreased [F-18]FDG uptake in medial and lateral temporal lobes and with increased level of [F-18]FDDNP binding in both areas; transaxial images at the level of hippocampus.

FIG. 6 is a set of in vitro digital autoradiography obtained using [F-18]MPPF (A, B) and with [F-18]FDDNP (C, D) on coronal whole-hemisphere brain tissue slices from a control subject (A, C) and from an AD patient (B, D); note the apparent decrease of [F-18]MPPF signal density in hippocampus (HIP, red arrow) and in the outer layer of gray matter on AD tissue (B); [F-18]FDDNP signal density is uniformly low on the control tissue (C) but is increased in the gray matter of temporal and limbic lobes compared to the other areas on the AD tissue (D).

### DESCRIPTION OF THE INVENTION

For convenience, the following abbreviations are used throughout the specification: AD, Alzheimer's disease; PET, positron-emission tomography; [F-18]FDG, 2-deoxy-2-[F-18]fluoro-D-glucose; [F-18]MPPF, 4-[F-18]fluoro-N-{2-[1-(2-methoxyphenyl)-piperazinyl]ethyl}-N-(2-pyridinyl)benzamide; [F-18]FDDNP, 2-(1-{6-[(2-[F-18]fluoroethyl)(methyl)amino]-2-naphthyl}ethylidene)malononitrile; NFT, neurofibrillary tangle; SP, β-amyloid senile plaque; DVR, relative distribution volume; BP, binding potential; SUVR, relative standardized uptake value; MMSE, Mini Mental State Examination; MCI, mild cognitive impairment; 5-HT_{1A}, serotonin 1A; ROI, region of interest; MTL, medial temporal lobe; LTL, lateral temporal lobe; PCG, posterior cingulate gyrus.

The present invention provides a method for detecting and monitoring Alzheimer's disease and related forms of dementia. In one embodiment of the invention, a radiolabeled, serotonin 5-HT_{1A} receptor-specific tracer is administered to a subject; one or more images of the subject's brain are created using PET or SPECT; and neuronal cell loss—and the existence or progression of dementia—is detected by comparing the image(s) to a control (e.g., a cognitively normal, age-matched human) or a prior image of the subject's brain.

Tracers considered to be useful in the practice of the invention include radiolabeled compounds that are suitable for use in PET or SPECT and have either an agonistic or antagonistic effect on 5-HT_{1A} receptors; that is, radiolabeled 5-HT_{1A} receptor agonists, as well as 5-HT_{1A} antagonists, can be used. The half-lifes and established behavior of [F-18], [C-11], and [I-123] radioisotopes makes them particularly preferred for use in practicing the present invention.

A number of ⁵-HT_{1A} receptor agonists and antagonists are known. A nonlimiting list of such compounds, radiolabeled analogs, and their syntheses, is provided below. Each of the cited references is incorporated by reference herein as if set forth in its entirety.

a. WAY-100635: N-{2-[4-(2-methoxyphenyl)-1-piperazinyl]ethyl}-N-(2-pyridinyl)cyclohexanecarboxamide;

b. “[carbonyl-C-11]-WAY-100635” (also known as “CWAY-100635”)—prepared by reacting [carbonyl-C11]cyclohexanecarboxylic acid chloride with 2-{2-[4-(2-methoxyphenyl)piperazinyl]ethyl} aminopyridine. Hwang D-R, Simpson N R, Montoya J, Mann J J, Laruelle M (1999). An improved one-pot procedure for the preparation of [11C-carbonyl]-WAY100635. Nucl. Med. Biol. 26, 721-727;

c. “[carbonyl-C-11]desmethyl-WAY-100635”—prepared by reacting [carbonyl-C11]cyclohexanecarboxylic acid chloride with 2-{2-[4-(2-hydroxyphenyl)piperazinyl]ethyl} aminopyridine. (Maiti D K, Chakraborty P K, Chugani D C, Muzik O, Mangner T J, Chugani H T (2005). Synthesis procedure for routine production of [carbonyl-C11]desmethyl-WAY-100635. Appl. Radiat. Isotop. 62, 721-727.);

d. “FCWAY”: N-{2-[4-(2-methoxyphenyl)-1-piperazinyl]ethyl}-N-(2-pyridinyl)-trans-4-fluorocyclohexanecarboxamide;

e. “[F-18]-FCWAY”—prepared by reacting pentafluorobenzyl trans-4-[F-18]fluorocyclohexanecarboxylate with 2-{2-[4-(2-methoxyphenyl)piperazinyl]ethyl} minopyridine. Lang L, Jagoda E, Schmall B, Vuong B-K, Adams H R, Nelson A L, Carson R E, Eckelman W C. (1999) Development of fluorine-18-labeled 5-HT_{1A} antagonists. J. Med. Chem. 42, 1576-1586;

f. MPPF: N-{2-[4-(2-methoxyphenyl)-1-piperazinyl]ethyl}-N-(2-pyridinyl)-4-fluorobenzamide;

g. “[F-18]-MPPF”—prepared as described in Le Bars, D.; Lemaire, C.; Ginovart, N.; Plenevaux, A.; Aerts, J.; Brihaye, C.; Hassoun, W.; Leviel, V.; Mekhsian, P.; Weissmann, D.; Pujol, J. F.; Luxen, A.; Comar, D. (1998), High-yield radiosynthesis and preliminary in vivo evaluation of p-[F-18]MPPF, a fluoro analog of WAY-100635. Nucl. Med. Biol. 25, 343-350;

h. “NAD-299”: (R)-3-N,N-dicyclobutylamino-8-fluoro-3,4-dihydro-2H-1-benzopyran-5-carboxamide (sometimes referred to as “robalzotan”); and

i. “[C-11]NAD-299”—prepared as described in Sandel, J., Halldin, C., Hall, H., Thorberg, S, Werner, T., Sohn, D., Sedvall, G., Farde, L., Radiosynthesis and Autoradiographic Evaluation of [C-11]NAD-299, a Radioligand for Visualization of the 5-HT_{1A} Receptor, Nucl. Med. Biol. 26, 159-164 (1999). The [F-18]-radiolabeled analog of NAD-299 also should be a useful tracer;

It is predicted that [F-18]- and [C-11]-radiolabeled analogs of “[H-3]8-OH-DPAT” ([³H]-8-hydroxy-2-(di-n-propylamino)tetraline)) also should work.

Also included are radiolabeled compounds suitable for use in SPECT and having an agonistic of antagonist effect on 5-HT_{1A} receptors. A nonlimiting example is MPPI, a derivative of MPPF which carries iodine 123 instead of fluorine. Kung, H. F., Frederick, D., Kim, H. J., McElgin, W., Kung, M. P., Mu, M., Mozley, P. D., Vessotskie, J. M., Stevenson, D. A., Kushner, S. A., and Zhuang, Z. P. (1996). In vivo SPECT imaging of 5-HT_{1A} receptors with [123I]p-MPPI in nonhuman primates. Synapse 24, 273-281.

Preferably, the tracer is administered via intravenous injection.

In one embodiment of the invention, neuronal cell loss associated with dementia in a subject is quantitatively evaluated, in vivo, in the following manner: a radiolabeled, 5-HT_{1A} receptor-specific tracer is administered to the subject; a dynamic data set corresponding to radioactivity in the subject's brain is generated using PET or SPECT; a parametric data set is generated from the dynamic data set; a set of regions-of-interest (ROIs) in the subject's brain are identified; the parametric data set is used to determine tracer binding potential values for the set of ROIs; and the determined tracer binding potential values are compared with tracer binding potential values obtained from a prior PET or SPECT scan of the subject, or a PET or SPECT scan of an age-matched, cognitively normal control. Advantageously, the steps can be repeated two or more times. Indeed, in one aspect of the invention, the progression (or absence) of AD or other dementia in the subject is monitored on an ongoing basis by repeating the steps at substantially regular intervals, for example, twice weekly, weekly, twice monthly, monthly, twice quarterly, quarterly, twice annually, anually, every three years, every five years, every ten years, or even longer (or shorter) intervals.

In one embodiment of the invention, the parametric data set is generated using Logan plot analysis. Alternatively, the parametric data set is generated using tracer kinetic modeling.

The ROIs are identified by comparing a PET or SPECT image of the subject's brain with an MRI of the subject's brain, and selecting one or more desired anatomical regions. In general, it is preferred to select ROIs from one or more neo-cortical regions, and/or the dorsal raphe nucleus of the subject's brain, the sites where large pyramidal neurons are found. Of particular interest are either or both hippocampi, the medial temporal lobe, the cingulate cortex, the insular cortex, and the dorsal raphe nucleus. Alternatively, the regions of interest are identified by examining one or more PET or SPECT images of the subject's brain and identifying areas of apparent tracer uptake.

### EXAMPLES

### Subjects and Clinical Assessments

Nineteen subjects were recruited through referrals from the UCLA Alzheimer's Disease Center, the UCLA Neuropsychiatric Institute Memory and Aging Research Center, and private referrals and were part of a larger PET imaging study in AD. Written informed consent was obtained in accordance with the procedures set by the Human Subjects Protection Committee, University of California at Los Angeles. None of the subjects had a history of other neurological, medical, or psychiatric condition and all were free from selective serotonin re-uptake inhibitors, beta-blockers (e.g. pindolol) or anti-anxiety drugs, with known effect on 5-HT_{1A} receptors. They all received neurological, psychiatric, and neuropsychological evaluations; an MRI scan; three PET scans ([F-18]MPPF, [F-18]FDG, and [F-18]FDDNP), and routine laboratory tests. APOE genotyping was performed on sixteen subjects who gave consent for it. Clinical diagnoses were made with investigators blind to genetic data; image data were analyzed; and ROIs determined with investigators blind to clinical and genetic findings. Results of Mini Mental State Exam were used as a measure of global cognitive decline in AD (1) and Buschke-Fuld Selective Reminding Test (Total Recall), a word list learning task (2).

Eight subjects (five females and three males; 5F/3M) met diagnostic criteria of dementia of the Alzheimer type, six subjects (2F/4M) met criteria of mild cognitive impairment, and five subjects (2F/3M) were cognitively normal controls. Four AD subjects, three MCI subjects, and three control subjects were APOE-ε4 carriers (six ADs: two ε3/ε3, three ε3/ε4, and one ε4/ε4 five; five MCIs: two ε3/ε3, two ε3/ε4, and one ε4/ε4; controls: two ε3/ε3, and three ε3/ε4 six). Ages and education of the cohort (average±SD) were as follows: AD—age 79.0±7.8 years, education 16.3±3.1 years; MCI—age 72.0±13.9 years, education 16.2±3.9 years; Controls—age 61.2±8.6 years, education 15.7±3.5 years). Differences in mean ages between groups was due to an increased attrition rate in control and MCI groups due to the length and complexity of the study protocols requiring multiple scans as well as cognitive testing (See below). However, it has been earlier shown that even though serotonin 1A receptor densities in brain could be affected by age, no age-dependent differences have been observed in subjects older than 60 years. (3)

Acquisition Protocols

All radiofluorinated imaging probes were prepared at very high specific activity (>37 GBq/μmol) by nucleophilic [F-18]fluorination using described procedures ([F-18]MPPF (4); [F-18]FDDNP (5); [F-18]FDG (6)).

All scans were performed with ECAT HR or ECAT HR+ scanners (Siemens-CTI, Knoxville, Tenn.). The subjects were in the supine position with the imaging plane parallel to the orbito-meatal line and with their eyes open and ears unoccluded. A 20 min transmission scan was performed to correct PET data for radiation attenuation. 320-550 MBq of a PET tracer was injected as a bolus injection via the in-dwelling venous catheter, and the consecutive dynamic head PET scans were performed for 1 hour with [F-18]FDG or for 2 hours with [F-18]MPPF or with [F-18]FDDNP.

All PET scans were decay corrected and reconstructed using filtered-back projection (Hann filter, 5 mm FWHM) with scatter and attenuation correction. The resulting images contained either 47 contiguous slices with 3.4 mm plane-to-pane separation (EXACT HR) or 63 contiguous slices with the plane-to-plane separation of 2.42 mm (EXACT HR+).

Anatomical brain magnetic resonance scans were obtained using either a 1.5 Tesla magnet (General Electric-Signa, Milwaukee, Wis.) or a 3 Tesla magnet (General Electric-Signa) scanner. Thirty-six transaxial planes were collected throughout the brain volume, superior to the cerebellum. A double echo, fast spin echo series using a 24-cm field of view and 256×256 matrix with 3 mm/0 gap (TR=6000 [3T] and 2000 [1.5T]; TE=17/85 [3T] and 30/90 [1.5T]) An inter-modality image co-registration program (7) that uses image segmentation and simulation as preprocessing procedures was used to co-register PET and anatomical MRI images of each subject. Rules for ROI drawing were based on the identification of gyral and sulcal landmarks with respect to the atlas of Talairach and Tournoux (8).

Quantitative PET Data Analysis

Quantitation of [F-18]MPPF binding was performed using Logan plot graphical analysis, using the cerebellum, an area largely devoid of 5-HT_{1A} receptors, as the reference region (9). The Logan plot is linear between 15-120 minutes post-injection with a slope proportional to distribution volume ratio (DVR=Bₘₐₓ/K_{d}+1). Tracer binding potential (BP=Bₘₐₓ/K_{d}) parametric images were analyzed using ROIs drawn bilaterally on hippocampus, lateral temporal lobe, parietal lobe, frontal lobe, and posterior cingulate cortex, and a single ROI on the dorsal raphe nucleus using MRI as a guide. The extracted BP values are shown in Table 1. Total binding potentials (BPT, BPT=BP×volume) were determined for left and right hippocampus by multiplying the hippocampal BP values with the hippocampus volumes on co-registered MRI.

Quantitation of [F-18]FDDNP data was performed using the Logan plot graphical analysis with the cerebellum as the reference region. Distribution volume ratio (DVR) parametric images were created as described elsewhere. ROIs were drawn bilaterally on the early frames of the dynamic PET scan (3 min to 6 min post-injection) using MRI as a guide bilaterally on the transaxial images on frontal lobe, on parietal lobe, on medial temporal lobe, and on lateral temporal lobe. In addition, a single ROI was placed on posterior cingulated gyrus. DV values for cortical regions were extracted from the [F-18]FDDNP DV parametric image using the above-described ROI set. DV values were determined for all 9 cortical ROIs and for cerebellum. Relative global neocortical [F-18]FDDNP distribution volume (global DVR) was determined by dividing the DV average value of the eight neocortical ROIs with the DV average value of cerebellar ROIs. In similar fashion, regional relative distribution volumes (DVRs) were determined for frontal, parietal, lateral temporal and medial temporal areas by dividing the DV average of both ROIs from the corresponding region with the DV average value of cerebellar ROIs. The posterior cingulated gyrus DVR was determined by dividing the cingulated gyrus DV value with the DV average value of cerebellar ROIs.

The dynamic [F-18]FDG PET images were summed (frames 30-60min) and the resulting image was used for the following analysis: ROIs were drawn bilaterally on transaxially oriented images on the gray matter signal in the frontal lobe, parietal lobe, lateral temporal lobe, and medial temporal lobe. In addition, one ROI was drawn on the posterior cingulated gyrus. ROIs were also drawn bilaterally on the motor cortex as a reference region. Values for each ROI were extracted and relative standardized uptake values were calculated for each region separately. The average of both ROIs from the same region was divided by the average of ROI values for the motor cortex. In a similar way, the ROI value for the posterior cingulated gyrus was normalized by the average value for motor cortex. Finally the global [F-18]FDG SUVR index was calculated for the parietal, medial temporal, and lateral temporal lobes.

[F-18]MPPF PET Scans, Imaging Resolution and Cerebral Atrophy in AD

Underestimation of cortical activity with PET due to partial volume effects (i.e. averaging with surrounding structures) resulting from cortical atrophy in AD is an important variable that has been earlier recognized for [F-18]FDG (10). Accordingly, partial volume effects on PET data resulting from 5-HT1A ligands were considered based on the atrophy observed in AD (11). The hippocampus is the brain area with the highest level of 5-HT1A receptors. In aged-matched controls, the linear dimension of the coronal cross-section of the hippocampus is about one cm, with a total volume of 3.40±0.52 cm³ (12). For a spatial resolution of approximately 5 mm FWHM for the scanner we used in this work (ECAT HR+tomograph (Siemens-CTI, Knoxville, Tenn.)) and a cylindrical shape object of this size coupled with a coronal ROI size of 1 cm diameter, the partial volume effect has a recovery coefficient of ˜0.69 (13).

For AD subjects with atrophic hippocampus, the hippocampal volume is reduced by an average of 30% as compared to age-matched control with reduction in the linear dimension of about 15% (assuming no lengthwise reduction) that will give a recovery coefficient of about 0.55 for a object size matched ROI. Since the recovery coefficient is applied to the activity above the background level, that is around 0.5 ml/g, the extra partial volume effect due to the atrophic hippocampus in AD subjects (compared with age-matched controls) on the hippocampal ROI value is thus expected to be less than 14% (=((1.5−0.5)/0.69*0.55+0.5)/1.5), which is less than half the average amount of reduction observed in this study for the hippocampal ROI in AD compared to that in normal controls. Similarly, application of regional MRI-based partial volume corrections has suggested that regional cortical hypometabolic changes measured with [F-18]FDG PET cannot account for the metabolic differences between AD patients and age-matched control subjects (14, 15).

Statistical Analysis

Nonparametric analyses of variance were conducted to assess whether there were significant differences in hippocampus [F-18]MPPF BP, [F-18]MPPF BPT or volume between AD, MCI and control groups, controlling for age. Spearman rank correlations (rₛ) were used to determine the correlation between hippocampus [F-18]MPPF BP, [F-18]MPPF BPT or volume and FDG-PET/FDDNP-PET/neuropsychological measures.

In vitro Procedures with Brain Specimens

Two AD and three control brain samples were obtained at the time of autopsy from the Alzheimer's Disease Research Center at USC Keck School of Medicine, Los Angeles. Glass mounted whole-hemisphere brain cryosections (100 μm thick), cut at the level of hippocampus, were incubated with 1.85 MBq/mL of [F-18]MWPF in 50 mM Tris buffer/saline (pH=7.4) for 2 hours at room temperature. The samples were exposed to β⁺-sensitive phosphor storage plates for 60 min, scanned in with BAS 5000 Phosphorimager, and analyzed with MacBAS software provided with the instrument (Fuji Film Medical Systems USA, Stamford, Conn.). ROIs were drawn on inner and outer bands of the gray matter of inferior temporal gyrus and on CA1 field of hippocampus. All values were normalized to the inner layer, known to be well preserved in AD (16). [F-18]FDDNP autoradiography was performed as described elsewhere (17). In brief, the de-fattened tissue slices were incubated with 0.37 MBq/mL of [F-18]FDDNP in 1% ethanol in normal saline for 25 min at room temperature followed by differentiation in 60% 2-methyl-2-butanol (3 min). The sections were exposed to β⁺-sensitive phosphor storage plates for 60 min and scanned in with BAS 5000 Phosphorimager.

Results

In vivo Imaging Experiments

Quantification of [F-18]MPPF Serotonin 1A Receptor Binding

Results of the [F-18]MPPF quantitative analysis (binding potential, BP) for different brain regions in AD, MCI and control groups are shown in Table 1. Quantification of [F-18]MPPF PET data based on Logan plot graphical analysis with the cerebellum as a reference region was tested in healthy volunteers, and BP was confirmed to be a good index of local receptor density (Bₘₐₓ) (18). Inter-group comparison revealed 27% drop in hippocampus BP values in AD subjects when compared to control subjects (mean±SD: AD 1.18±0.26 vs. control 1.62±0.07; P<0.001) and 41% drop in dorsal raphe nucleus BP values (AD 0.37±0.20 vs. control 0.63±0.09; P<0.01). No other area had BP values statistically significantly different between the groups.

TABLE 1
Results of [F-18]MPPF PET quantitative data analysis.

| | | | | | | Posterior |
|---|---|---|---|---|---|---|
| | | | | | Lateral Temporal | Cingulate |
| Hippocampus | | Raphe Nuclei | Frontal Lobe | Parietal Lobe | Lobe | Gyrus |
| Control | 1.62 ± 0.07 | 0.63 ± 0.09 | 0.49 ± 0.15 | 0.63 ± 0.15 | 0.82 ± 0.17 | 0.68 ± 0.06 |
| MCI | 1.41 ± 0.14* | 0.52 ± 0.11 | 0.44 ± 0.12 | 0.43 ± 0.11 | 0.80 ± 0.11 | 0.56 ± 0.12 |
| AD | 1.18 ± 0.27^{‡} | 0.37 ± 0.20^{†} | 0.41 ± 0.13 | 0.48 ± 0.13 | 0.73 ± 0.16 | 0.56 ± 0.20 |

MPPF results are given as mean BP ± 1 SD.
Statistical significance of separation from the control group (ANOVA):
*P < 0.05,
^{†}P < 0.01,
^{‡}P < 0.001.

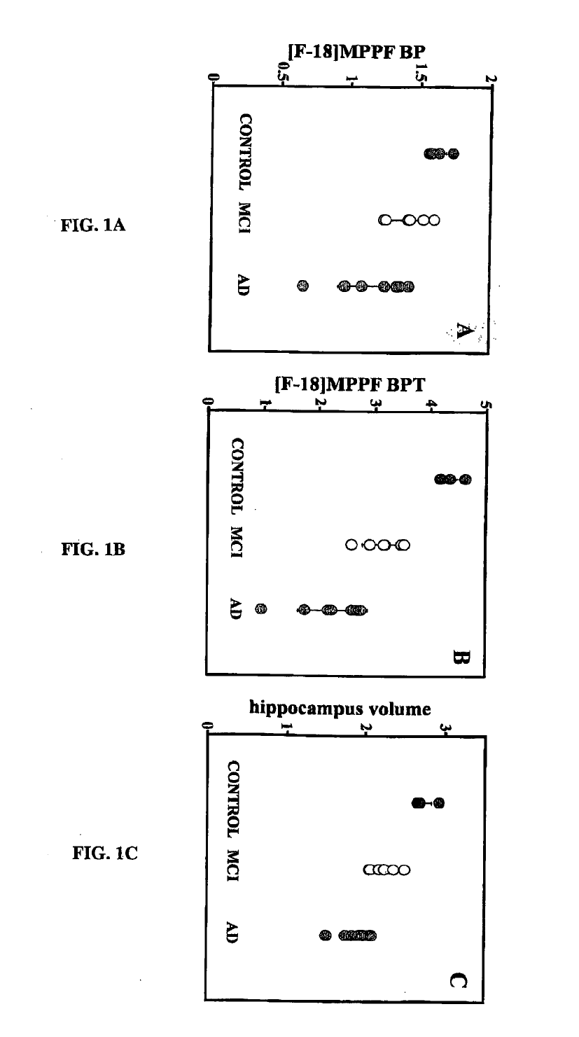

FIG. 1A demonstrates variations in hippocampus BP in all three groups. In AD hippocampus BP values ranged between 0.65 and 1.25, reflecting the heterogeneous nature of this group, which had large variation in disease severity (MMSE scores ranged between 8 and 27). In the group of MCI subjects, the mean hippocampus BP value was 13% lower than in controls (1.41±0.14; P<0.05). BP values in all other analyzed areas were not different from controls. When, in addition to decreasing receptor density, one takes in account also hippocampus volume losses starting at early AD stages, it becomes obvious that the product of both variables, total number of 5-HT_{1A} receptors (total binding potential, BPT=BP×volume), should be affected much more drastically than any of the components by itself. Indeed, the observed hippocampus BPT values (FIG. 1B) were lower for 24% for MCIs and for 49% for ADs when compared with controls (controls 4.40±0.22; MCI 3.14±0.34 (P<0.05); AD 2.24±0.61 (P<0.0001)).

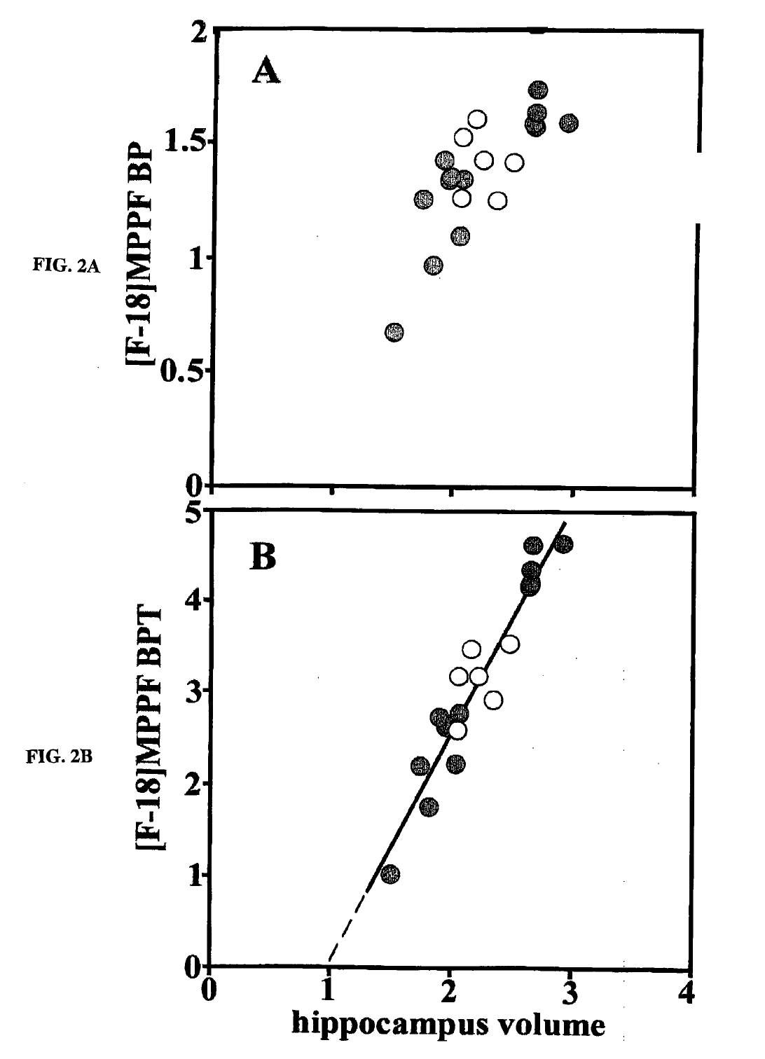

The hippocampus volumes measured in these patient populations (controls 2.71±0.12 cm³; MCI 2.23±0.18 cm³ (P<0.05); AD 1.88±0.19 cm³ (P<0.0001)) reveal volume decreases of 18% in MCIs and 31% in ADs (FIG. 1C), and are in excellent agreement with earlier reports (9). Hippocampus volume values are also correlated with [F-18]MPPF BP (FIG. 2A, Spearman's r=0.76, P=0.0002) and with [F-18]MPPF BPT (FIG. 2B; Spearman's r=0.95, P<0.0001) the hippocampus volume with extrapolated [F-18]MPPF BPT=0 almost 1.00 cm³, which is close to 1.06 cm³, a value determined post-mortem in very severe, immobile AD patients with the most severe hippocampus atrophy (19).

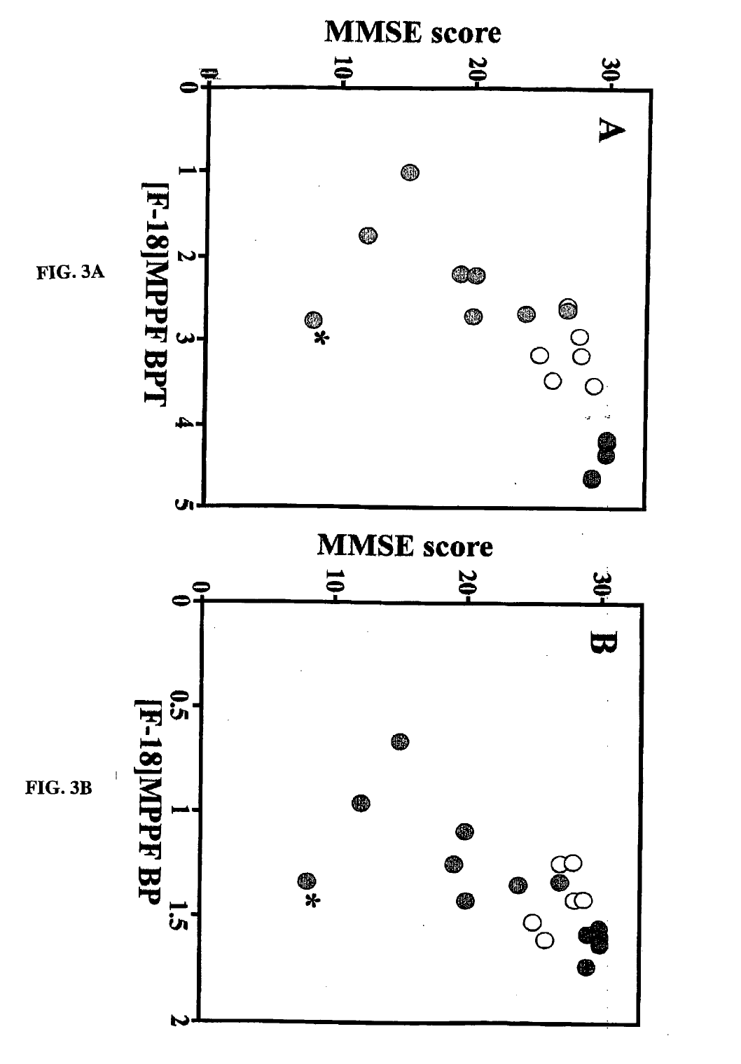

The hippocampus is the part of the medial temporal lobe system responsible for declarative memory; therefore, decreasing [F-18]MPPF BP and [F-18]MPPF BPT values in our groups correlate well with progressive cognitive decline (MMSE scores; controls 29.6±0.50; MCI 27.2±1.50 (P<0.01); AD 18.1±6.2 (P<0.005)) as shown in the FIGS. 3A and 3B (Spearman's r=0.67, P=0.0015; Spearman's r=0.82, P<0.0001; respectively). Since the MMSE test is targeting all aspects of cognition, the earliest memory problems observed in MCI and early AD do not have a big impact on the MMSE scores, which may explain its apparent lack of sensitivity for separation of MCIs and controls despite large [F-18]MPPF BP and [F-18]MPPF BPT decrease. This became obvious when the results of Buschke-Fuld Selective Reminding Test (Total Recall), a word list learning task (2), were correlated with [F-18]MPPF BPT values for controls and MCIs (Spearman's r=0.709, P<0.015). By itself Buschke test separated well MCIs from controls (controls 114.0±19.0; MCI 69.5±15.7 (P<0.005)), but it is too hard to complete for AD patients.

[F-18]FDG PET Analysis

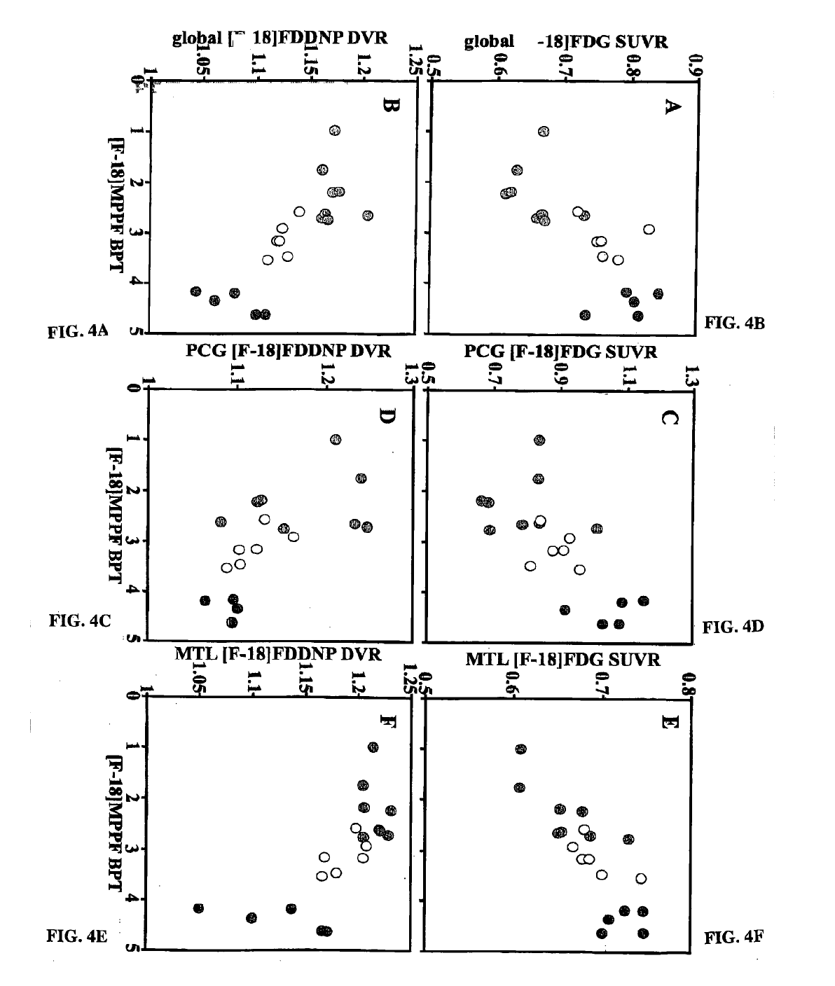

Disconnection of various neuronal circuits, and the neuronal loss behind it, profoundly change patterns of the brain energy consumption and glucose metabolism as the primary energy substrate in the brain in AD. [F-18]FDG SUVR values shown in Table 2 (normalized to motor strip, an area with relatively preserved glucose utilization) are indicative of that fact. As expected for the symptomatic AD patients, we have observed significant [F-18]FDG SUVR decreases in all analyzed areas in our AD group. An average of parietal lobe, LTL and MTL [F-18]FDG SUVRs was used as an index of global [F-18]FDG uptake for easier comparison. The pattern of decreased glucose uptake in early presymptomatic AD shows that posterior cingulate gyrus (PCG) is the area with the earliest decline (20, 21). Consistently with these reports we have found significant decrease for the MCI group only in PCG SUVR (controls 1.05±0.09; MCI 0.89±0.06 (P<0.01)). Indication that the neuronal damage in medial temporal lobe could cause the functional loss in PCG comes from lesioning experiments in baboons, in which lesions limited to entorhinal and perirhinal cortices caused decrease of [F-18]FDG uptake in several brain region including the PCG (22). Comparisons of [F-18]MPPF BPT values with the global, PCG and MTL [F-18]FDG SUVR values in the same subjects show that these measures are correlated (FIG. 4A-4C) (Sperman's r coefficients, P values: global r=0.80, P<0.0001; PCG r=0.73, P=0.0004; MTL r=0.81, P<0.0001).

TABLE 2
Results of [F-18]FDG PET and [F-18]FDDNP PET quantitative data analysis

| | | | | | | | Posterior |
|---|---|---|---|---|---|---|---|
| | | | | | Medial Temporal | Lateral Temporal | Cingulate |
| | | Global | Frontal Lobe | Parietal Lobe | Lobe | Lobe | Gyrus |
| FDG | CTRL | 0.80 ± 0.04 | 0.95 ± 0.08 | 0.89 ± 0.06 | 0.73 ± 0.02 | 0.86 ± 0.06 | 1.05 ± 0.09 |
| | MCI | 0.77 ± 0.04 | 0.90 ± 0.02 | 0.84 ± 0.05 | 0.69 ± 0.03* | 0.81 ± 0.05 | 0.89 ± 0.06^{†} |
| | AD | 0.66 ± 0.04^{§} | 0.79 ± 0.07^{†} | 0.67 ± 0.09^{§} | 0.66 ± 0.04^{‡} | 0.66 ± 0.07^{§} | 0.79 ± 0.11^{§} |
| FDDNP | CTRL | 1.08 ± 0.03 | 1.05 ± 0.01 | 1.07 ± 0.03 | 1.12 ± 0.05 | 1.07 ± 0.03 | 1.07 ± 0.05 |
| | MCI | 1.12 ± 0.01* | 1.08 ± 0.01^{‡} | 1.09 ± 0.03 | 1.19 ± 0.02* | 1.13 ± 0.03* | 1.12 ± 0.03 |
| | AD | 1.17 ± 0.01^{§} | 1.11 ± 0.03^{§} | 1.17 ± 0.03^{§} | 1.23 ± 0.04^{†} | 1.17 ± 0.03^{§} | 1.18 ± 0.06^{†} |

FDG results are given as mean SUVR ± 1 SD;
FDDNP results are given as mean DVR ± 1 SD.
Statistical significance of separation from the control group (ANOVA):
*P < 0.05,
^{†}P < 0.01,
^{‡}P < 0.005,
^{§}P < 0.001.

[F-18]FDDNP PET Analysis

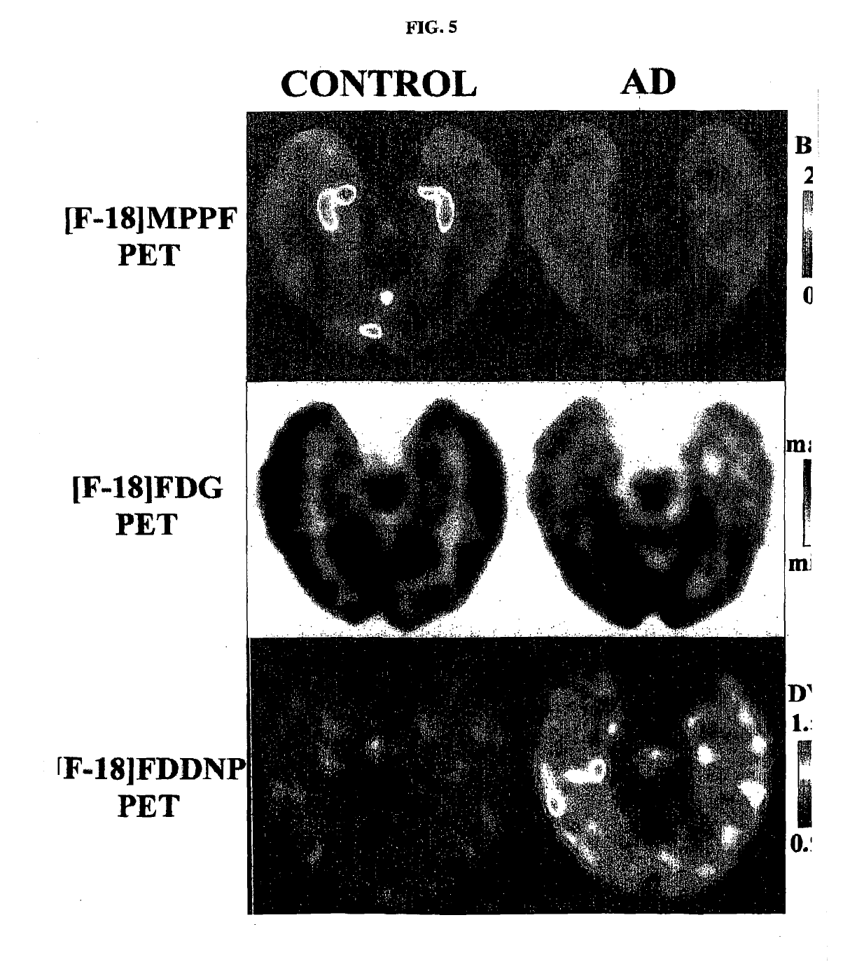

In vivo assessment of the extent and pattern of neuropathological load (NFTs and SPs) was performed with [F-18]FDDNP PET (Table 2). The AD group had DVR values significantly elevated in all areas analyzed when compared with the control group. This was to be expected in symptomatic AD patients, with symptoms indicative of damage in neuronal circuits and progression of pathology (NFTs and SPs) spread beyond the medial temporal lobe structures (Braak NFT stages V and VI and Braak SP stages B and C) (23). For easier comparison, global relative [F-18]FDDNP distribution volume was calculated by averaging the values for frontal, parietal, medial temporal and lateral temporal lobe DVRs for each subject. The mean global [F-18]FDDNP DVR value for the AD group was 1.17±0.01 and it was significantly elevated when compared with the mean global [F-18]FDDNP DVR value for the control group (1.08±0.03, P<0.0005). Several MCI group regional [F-18]FDDNP DVR measures were also significantly different from the control group [F-18]FDDNP DVR values in the same areas (frontal lobe, LTL, MTL). Correlations of [F-18]MPPF BPT in hippocampus with global, PCG and MTL [F-18]FDDNP DVR values are shown in FIGS. 4D-4E (Sperman's r coefficients, P values: global r=−0.86, P<0.0001; PCG r=−0.71, P=0.0007; MTL r=−0.61, P=0.006). As an illustrative example, transaxial views at the level hippocampus of [F-18]MPPF BP parametric images, of a summed [F-18]FDG image, and of [F-18]FDDNP DVR parametric images for a non-demented control subject and for one of the severely affected AD patients are shown in FIG. 5.

In vitro [F-18]MPPF and [F-18]FDDNP Digital Autoradiography

The quantitative results of regional [F-18]MPPF binding density from the digital autoradiographs obtained on two AD and three control tissue slices show decreased binding ratio (BR) in hippocampus CA1 filed/subiculum (relative to the inner layer of inferior temporal gyrus gray matter, known from the literature to be well preserved in AD (16)). The hippocampal BR values from AD samples were 1.03 and 1.18 and from control samples 3.29, 3.00, and 2.98. This represents a 60-70% drop in in vitro binding and agrees with general declines in [F-18]MPPF BP (and BPT) values obtained with living AD patients (Table 2). The [F-18]MPPF binding density is also decreased in the outer layers of gray matter in the inferior temporal gyrus in both AD cases (BR=1.13 and 1.16) when compared with non-demented cases (BR=2.01, 1.46 and 1.40).

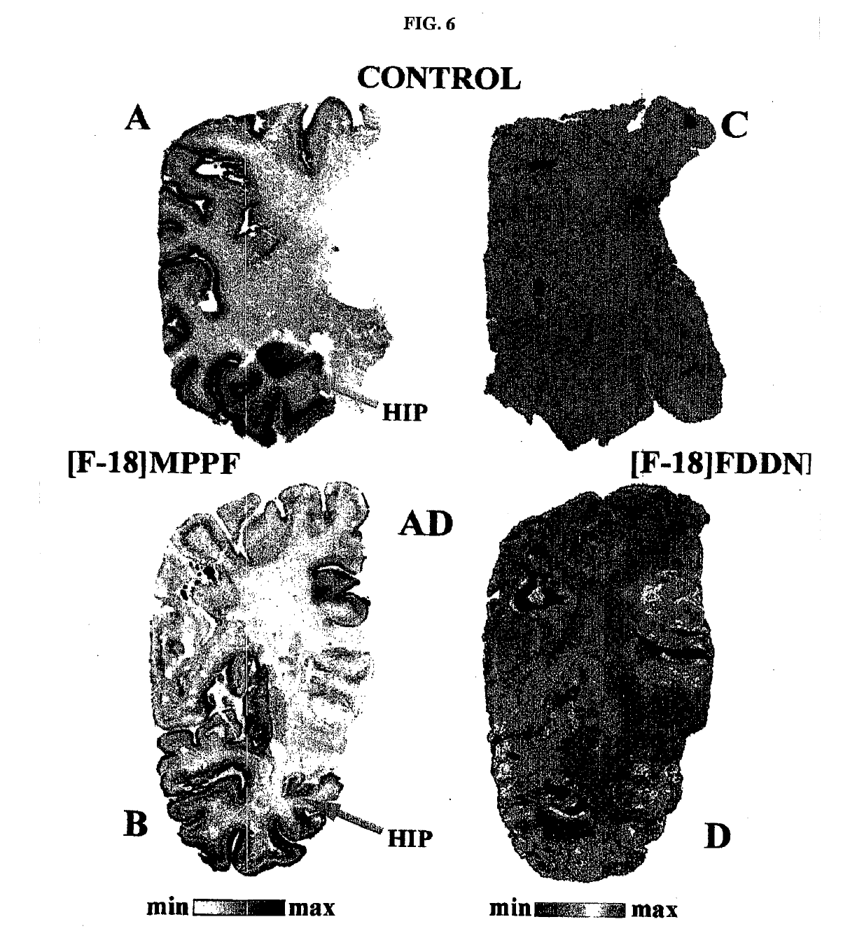

AD tissue also shows a pattern of strong [F-18]FDDNP binding to the gray matter in the temporal lobe and in cingulated gyrus, and less prominent binding in the rest of gray matter. Tau aggregates are mainly found in the hippocampus and in the medial temporal lobe cortex, whereas amyloid plaques are the main pathology labeled with FDDNP in the lateral temporal lobe and in the posterior cingulated gyrus. In contrast, none of the gray matter areas from the normal control brain samples showed [F-18]FDDNP binding above background levels in white matter. Representative autoradiograms are shown in FIG. 6.

The method has been tested on at human subjects, including healthy, cognitively normal controls, and moderately demented AD patients.

It will be appreciated that the methods for assessing neuronal cell loss described herein can also be used in conjunction with other vivo techniques for evaluating one or more additional characteristics of the subject, such as glucose metabolic activity, deposits of neurofibrillary tangles and/or senile plaques, and behavioral characteristics. For example, in one embodiment, one or more in vivo techniques for detecting amyloid or tau aggregates and/or monitoring regional decreases in glucose metabolism in parietal and temporal lobes are utilized. Nonlimiting examples of such techniques include [F-18]FDDNP PET, for detecting amyloid or tau aggregates (amyloid plaques and NFTs); FDG-PET (for monitoring regional decreases in glucose metabolism in parietal and temporal lobes); and similar techniques using other [F-18], [C-11], [I-123], or other suitable radiolabeled markers. Nonlimiting examples of behavioral characteristics include MMSE and Buschke scores In general, AD patients exhibit elevated FDDNP, low FDG, and low (50% or less) MPPF accumulation, i.e., low 5-HT_{1A} receptor density in the hippocampi—a clear indication of significant neuronal cell loss.

The invention has been described with reference to various embodiments and examples, but is not limited thereto. Variations may be made without departing from the invention's scope, which is limited only by the appended claims, which are to be afforded their full scope, both literally and by equivalents. The invention is limited only by the appended claims and their equivalents.

### REFERENCES

Each reference is incorporated herein as if set forth in its entirety.
1. Folstein, M. F., Folstein, S. E. & McHugh, P. R. (1975) J. Psychiatr. Res. 12, 189-198.
2. Buschke, H. & Fueld, P. A. (1974) Neurology 24, 1019-1025.
3. Parsey, R. V., Oquerndo, M. A., Simpson, N. R., Ogden, R. T., Van Heertum, R., Arango, V. & Mann, J. J. (2002) Brain Res. 954, 173-182.
4. Le Bars, D., Lemaire, C., Ginovart, N., Plenevaux, A., Aerts, J., Brihaye, C., Hassaoun, W., Leviel, V., Mekshian, P., Weissmann, D., Pujol, J. F., Luxen, A. & Comat, D. (1998) Nucl. Med. Biol. 25, 343-350.
5. Shoghi-Jadid, K., Small, G. W., Agdeppa, E. D. Kepe, V., Ercoli, L. M., Siddarth, P., Read, S., Satyamurthy, N., Petri\check{c}, A., Huang, S.-C. & Barrio. J. R. (2002) Am. J. Geriatr. Psychiatry 10, 24-35.
6. Padget, H. C., Schmidt, D. G., Luxen, A., Bida, G. T., Satyamurthy, N., & Barrio, J. R. (1989) Int. J. Rad. Appl. Instrum. [A] 40, 433-445.
7. Lin, K. P., Huang, S. C., Baxter, L. & Phelps, M. E. (1994) IEEE Trans. Nucl. Sci. 41, 2850-2855.
8. Talairach, J. & Tournoux, P. (1988) Coplanar Stereotaxic Atlas of the Human Brain. Three-Dimensional Proportional System: An Approach to Cerebral Imaging (Thieme, New York).
9. Logan, J., Fowler, J. S., Volkow, N. D., Wang, G.-J., Ding Y.-S. and Alexoff, D. L. (1996) J. Cereb. Blood Flow Metabol. 16, 834-840.
10. Meltzer, C. C., Zubieta, J. K., Links, J. M., Brakeman, P., Strumpf, M. J. & Frost, J. J. (1996) J. Cereb. Blood Flow Metabol. 16, 650-658.
11. Thompson, P. M., Hayashi, K. M., de Zubicaray, G., Janke, A. L., Rose, S. E., Semple, J., Herman, D., Hong, M. S., Dittmer, S. S., Doddrell, D. M., and Toga, A. W. (2003). J. Neurosci. 23, 994-1005.
12. Convit, A., de Leon. M. J., Tarshish, C., de Santi, S., Tsui, W., Rusinek, H., and George, A. (1997). Neurobiol. Aging 18, 131-138.
13. Hoffman, E. J., Huang, S.-C., and Phelps, M. E. (1979). J. Comput. Assist. Tomogr. 3, 299-308.
14. Meltzer, C. C., Zubieta, J. K., Brandt, J., Tune, L. E., Mayberg, H. S. & Frost, J. J. (1996) Neurology 47, 454-461.
15. Ibanez, V., Pietrini, P., Alexander. G. E., Furey, M. L., Teichberg, D., Rajapakse, J. C., Rapoport, S. I., Schapiro, M. B. & Horwitz, B. (1998) Neurology 50, 1585-1593.
16. Cross, A. J., Slater, P., Perry, E. K. & Perry, R. H. (1988) Neurochem. Int. 13, 89-96.
17. Agdeppa, A. D., Kepe, V., Petric, A., Satyamurthy, N., Liu, J., Huang, S. C., Small, G. W., Cole, G. M. & Barrio, J. R. (2003) Neuroscience 117, 723-730.
18. Costes, N., Merlet, I., Zimmer, L., Lavenne, F., Cinotti, L., Delforge, J., Luxen, A., Pujol, J.-F. & Le Bars, D. (2002) J. Cereb. Blood Flow Metab. 22, 753-765.
19. Bobinski, M., Wegiel, J., Wisniewski, H. M., Tarnawski, M., Bobinski, M., Reisberg, B., de Leon, M. J., & Miller, D. C. (1996) Neurobiol. Aging 17, 909-919.
20. Small, G. W., Ercoli, L. M., Silverman, D. H., Huang, S.-C., Komo, S., Bookheimer, S. Y., Lavretsky, H., Miller, K., Siddarth, P., Rasgon, N. L., Mazziota, J. C., Saxena, S., Wu, H.-M., Mega, M. S., Cummings, J. L., Saunders, A. M., Pericak-Vance M. A., Roses, A. D., Barrio, J. R. & Phelps, M. E. (2000) Proc. Natl. Acad. Sci. U.S.A. 97, 6037-6042.
21. Minoshima, S., Giordani, B., Berent, S., Frey, K. A., Foster, N. L. & Kuhl, D. E. (1997) Ann. Neurol. 42, 85-94.
22. Meguro, K., Blaizot, X., Kondoh, Y., Le Mestric, C., Baron, J. C. & Chavoix, C. (1999) Brain 122, 1519-1531.
23. Braak, H. & Braak, E. (1991) Acta Neuropathol. 82, 239-259.

What is claimed is:

1. A method for detecting or monitoring neuronal cell loss associated with dementia in a subject, in vivo, comprising:
administering a radiolabeled, serotonin 5-HT_{1A} receptor-specific tracer to the subject;
creating at least one image of the subject's brain using positron emission tomography (PET) or single-photon emission computed tomography (SPECT);
quantitating serotonin 5-HT_{1A} receptor density in an imaged region of the subject's brain; and
assessing neuronal cell loss associated with dementia by comparing the at least one image to a control or a prior image of the subject's brain.

2. A method as recited in claim 1, wherein the radiolabeled tracer comprises a 5-HT_{1A} agonist.

3. A method as recited in claim 1, wherein the radiolabeled tracer comprises a 5-HT_{1A} antagonist.

4. A method as recited in claim 1, wherein the radiolabeled tracer is selected from the group consisting of [carbonyl-C-11] WAY-100635, [carbonyl-C-11]desmethyl-WAY-100635, “[F-18]-FCWAY,” [F-18]-MPPF, [C-11]NAD-299, and MPPI.

5. A method as recited in claim 1, further comprising evaluating one or more additional characteristics of the subject, selected from the group consisting of glucose metabolic activity, deposits of neurofibrillary tangles and/or senile plaques, and behavioral characteristics.

6. A method as recited in claim 1, used in combination with one or more in vivo techniques for detecting amyloid or tau aggregates and/or monitoring regional decreases in glucose metabolism in parietal and temporal lobes.

7. A method as recited in claim 6, wherein said one or more in vivo techniques utilizes a [F-18] or [C-11] radiolabeled marker.

8. A method as recited in claim 6, wherein the subject's brain is additionally imaged using [F-18]-FDG-PET and/or [F-18]-FDDNP-PET or any other tracer useful for detecting amyloid or tau aggregates.

9. A method of quantitatively evaluating neuronal cell loss associated with dementia in a subject, in vivo, comprising:
(a) administering a radiolabeled, 5-HT_{1A} receptor-specific tracer to the subject;
(b) using positron-emission tomography (PET) or single-photon emission computed tomography (SPECT) to generate a dynamic data set corresponding to radioactivity in the subject's brain;
(c) generating a parametric data set from the dynamic data set;
(d) identifying a set of regions-of-interest in the subject's brain;
(e) using the parametric data set to determine tracer binding potential values for the set of regions-of-interest; and
(f) comparing the determined tracer binding potential values with tracer binding potential values obtained from (i) a prior PET or SPECT scan of the subject, or (ii) a PET or SPECT scan of an age-matched, cognitively normal control.

10. A method as recited in claim 9, wherein the radiolabeled tracer comprises a 5-HT_{1A} agonist.

11. A method as recited in claim 9, wherein the radiolabeled tracer comprises a 5-HT_{1A} antagonist.

12. A method as recited in claim 9, wherein the radiolabeled tracer is selected from the group consisting of CWAY-100635, [carbonyl-C-11]desmethyl-WAY-100635, “[F-18]-FCWAY,” [F-18]-MPPF, [C-11]NAD-299, and MPPI.

13. A method as recited in claim 9, wherein the parametric data set is generated using Logan plot analysis.

14. A method as recited in claim 9, wherein the parametric data set is generated using tracer kinetic modeling;

15. A method as recited in claim 9, wherein the regions of interest are located in one or more neo-cortical regions of the subject's brain.

16. A method as recited in claim 15, wherein the one or more neo-cortical regions are selected from either or both hippocampi, medial temporal lobe, cingulate cortex, insular cortex, and combinations thereof.

17. A method as recited in claim 9, wherein the regions of interest are located in the subject's dorsal raphe nucleus.

18. A method as recited in claim 9, wherein the regions of interest are located in at least one neo-cortical region of the subject's brain and in the subject's dorsal raphe nucleus.

19. A method as recited in claim 9, wherein the regions of interest are identified by comparing a PET or SPECT image of the subject's brain with a magnetic resonance image (MRI) of the subject's brain, and selecting one or more desired anatomical regions.

20. A method as recited in claim 9, wherein the regions of interest are identified by examining one or more PET or SPECT images of the subject's brain and identifying areas of apparent tracer uptake.

21. A method of quantitatively monitoring neuronal cell loss, in vivo, in a subject known or suspected to be suffering from dementia, comprising:
(a) administering a radiolabeled, 5-HT_{1A} receptor-specific tracer to the subject;
(b) using positron-emission tomography (PET) or single-photon emission computed tomography (SPECT) to generate a dynamic data set corresponding to radioactivity in the subject's brain;
(c) generating a parametric data set from the dynamic data set;
(d) identifying a set of regions-of-interest in the subject's brain;
(e) using the parametric data set to determine tracer binding potential values for the set of regions-of-interest; and
(f) comparing the determined tracer binding potential values with tracer binding potential values obtained from a prior PET or SPECT scan of the subject.

22. A method as recited in claim 21, wherein the dementia comprises Alzheimer's disease, frontal lobe dementia, or Lewy Body dementia.

23. A method as recited in claim 21, further comprising repeating steps (a)-(f) two or more times.

24. A method as recited in claim 21, further comprising repeating steps (a)-(f) on substantially regular intervals, selected from the group consisting of twice weekly, weekly, twice monthly, monthly, twice quarterly, quarterly, twice annually, anually, every three years, every five years, and every ten years.

25. A method for detecting or monitoring Alzheimer's disease in a subject, comprising:
administering a radiolabeled, serotonin 5-HT_{1A} receptor-specific tracer to the subject;
creating at least one image of the subject's brain using positron emission tomography (PET) or single-photon emission computed tomography (SPECT);
quantitating serotonin 5-HT_{1A} receptor density in an imaged region of the subject's brain; and
assessing existence or progression of Alzheimer's disease in the subject by comparing the image(s) to a control or a prior image of the subject's brain.
